(19) United States
(12) Patent Application Publication
(10) Pub. No.: US 2022/0269016 A1
(43) Pub. Date: Aug. 25, 2022

(54) CELL PRINTER COIL CONNECTOR

(71) Applicants: Igor Zlatkin, Mills, MA (US); Sidney Braginsky, Port Washington, NY (US); John C. Moore, Wrentham, MA (US)

(72) Inventors: Igor Zlatkin, Mills, MA (US); Sidney Braginsky, Port Washington, NY (US); John C. Moore, Wrentham, MA (US)

(73) Assignee: Digilab Inc., Hopkinton, MA (US)

(21) Appl. No.: 17/182,034

(22) Filed: Feb. 22, 2021

(51) Int. Cl. G02B 6/38

(52) U.S. Cl. CPC G02B 6/3887; G02B 6/3851; G02B 6/3879

(57) ABSTRACT

A unitary coiled connector component for use within a 3D bioprinter or cell printer. The unitary coiled connector comprising a metal sleeve, a PEEK insert, and a printer tip, and configured for connection to a valve within the cell printer. The unitary coiled connector being easily removable and replaceable to reduce repair time as well as reduce contamination to the cell printer during repair or replacement of the coiled connector. Also disclosed is an alternative embodiment of a printer block for retaining one or more unitary coiled connectors.

### CROSS REFERENCE TO RELATED APPLICATION

This application claims the benefit of U.S. Provisional Patent Application No. 62/981,449, filed on Feb. 25, 2020, the entire specification of which is incorporated by reference herein.

### FIELD OF THE DISCLOSURE

The field relates generally to regenerative medicine and tissue engineering, and more particularly to production of engineered tissues/organs having desired structures. The field of the disclosure also relates to bio-printers, and more specifically to an improved component of the printer for depositing cells in a microwell, a slide or plate, or upon a substrate or matrix.

### BACKGROUND

Preparing customizable live-cell arrays, tissue engineering, biofabrication/bioprinting, and related cell deposition applications have become increasingly popular. Microarray 3D bioprinting refers to dispensing very small amounts of cells along with other biological samples such as hydrogels, growth factors, extracellular matrices, biomolecules, drugs, DNAs, RNAs, viruses, bacteria, growth media, or combinations thereof, on a microwell/micropillar chip platform using a microarray spotter and then incubating the cells to create a mini-bioconstruct as part of the creation of relatively large scale tissue constructs that more accurately mimic the in vivo environment. This technology is revolutionizing tissue engineering and disease modeling for screening therapeutic drugs and studying toxicology. 3D-bioprinters or cell printers deposit one or more cells gently and with high efficiency upon a substrate or matrix. A challenge for tissue engineering is producing three-dimensional (3D), vascularized cellular constructs of clinically relevant size, shape, and structural integrity.

3D, live cell, and bio-printers are beneficial for numerous tasks, such as, for example, stem cell research and cancer biology, customized cell arrays/cell-based assays, automated cell dispensing for high-content analysis, cell-Cell interaction studies, cell-drug interaction studies (e.g. drug screens), cell culture in hydrogels, biofabrication/bioprinting research, tissue engineering, and regenerative medicine. The printing process is controlled by a computer according to a predetermined instruction, usually a computer-aided design (CAD) file of the respective tissues or object. The ultimate goal of the technology is to replicate functioning tissue and material, up to full organs, which then can be transplanted into human beings.

Live cell viability is an essential factor in cell printing, and higher viability is always desirable. The CellJet™ printer, commercially available from Digilab (Hopkinton, Mass.), incorporates a proprietary liquid dispensing technology, offering both on-the-fly and drop-by-drop non-contact cell printing while maintaining the highest viability of even the most delicate cells. 3D-Bioprinting is a natural combination of both 3D fine spatial printing and live-cell culturing. Cells are aspirated from their source through ceramic tips into coiled tubing and dispensed into microtiter plates or slides without making contact with any valve.

An integral component of the CellJet printer is the coiled tubing connector or adapter. The coiled tubing connector minimizes cell-sedimentation problems and ensures steady delivery of a close-to-constant average number of live cells per drop over the course of the experiment. The coiled tubing connector comprises a plastic coil that is connected on both ends to the printer during manufacture. One end of the coil comprises a metal sleeve, a PEEK (Polyether Ether Ketone) insert and a tip for printing, and the other end connects to a valve within the printer. The multiple components of the coiled tubing connector, however, create several potential issues, such as during cleaning, repairing and replacing.

The instant disclosure seeks to eliminate these issues via a component in which the tip, the PEEK insert, the metal sleeve, and the coil are integrated into a unitary or singular piece or component. Such a unitary component makes it easier to connect and disconnect the component from the printer and renders the component easier to repair or replace.

### BRIEF DESCRIPTION OF THE DRAWINGS

FIG. 1 illustrates exemplary coil connector components in accordance with the instant disclosure;

FIG. 2 is an exploded image of a commercially available coil connector component;

FIG. 3 illustrates a ferrule according to the instant disclosure;

FIG. 4 is an image depicting a commercially printing tip block; and

FIG. 5 depicts an alternative embodiment of the printing tip block in accordance with the instant disclosure.

Corresponding reference characters indicate corresponding parts throughout the drawings.

### DETAILED DESCRIPTION

Unless defined otherwise, all technical and scientific terms used herein have the same meaning as commonly understood by one of ordinary skill in the art to which the invention belongs.

All references to singular characteristics or limitations of the present disclosure shall include the corresponding plural characteristic or limitation, and vice versa, unless otherwise specified or clearly implied to the contrary by the context in which the reference is made.

Digilab Inc. manufactures an exemplary 3D (live cell) printer or bioprinter, the CellJet Cell Printer, which incorporates Digilab's proprietary liquid dispensing technology, offering both on-the-fly and drop-by-drop non-contact cell printing while maintaining the highest viability of even the most delicate cells. The CellJet Printer is a compact system fitting in most laboratory hoods and biological safety cabinets and which can be programmed with multiple different settings to fit the customer's needs and can be customized on demand. The CellJet Printer is equipped with a built-in digital camera (not shown) for enhanced control of the printing process and may optionally be supplied with a temperature-controlling block to print temperature-sensitive cells or matrices.

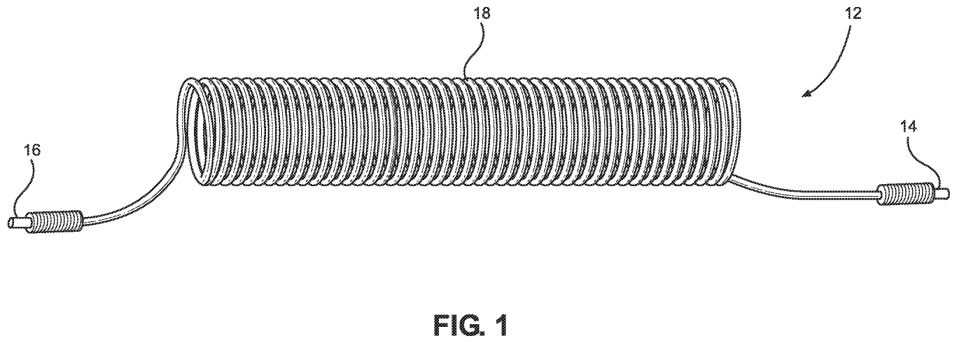

One integral component of the CellJet Printer is a coiled connector component 12 or adapter. An exemplary commercially available coiled connector 12 is illustrated in FIG. 1. The coiled connector minimizes cell-sedimentation problems and ensures steady delivery of close-to-constant average number of live cells per drop over the course of an experiment. In this disclosure, the term “connector” and “adapter” are used interchangeably and are intended to refer to the same thing.

Coiled connector 12 is typically fabricated from plastic and features a first lead end 14 and a second lead end 16. First and second lead end 14, 16 are typically straight (i.e., uncoiled) and spaced from each other by coiled section 18. Coiled connector 12 is not, however, limited to fabrication from plastic and may also be fabricated from silicone or any other inert material acceptable for use in medical devices and acceptable to the Food & Drug Administration (FDA) with regard to contamination, material breakdown and the like.

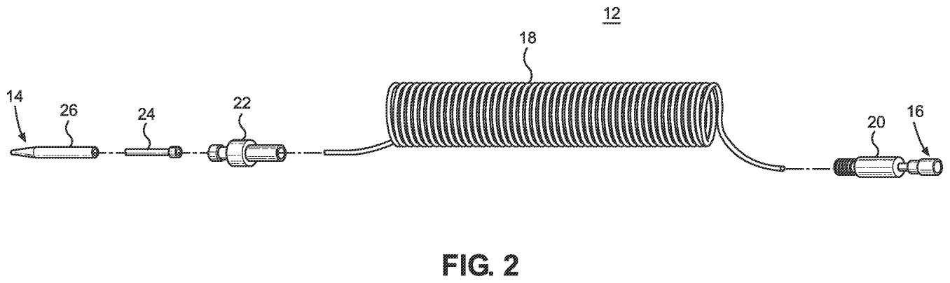

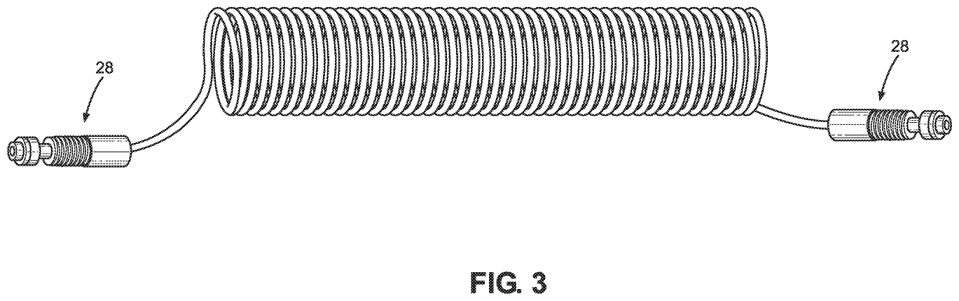

Referring to FIG. 2, a commercially available coiled connector 12 is depicted in an exploded configuration. As shown in FIG. 3, first lead end 14 of coiled connector 12 is attached to a valve 20, which is commonly a Lee valve. During printer operation, Lee valve 20 is located within a cell printer and is necessary for connecting coiled connector 12 within the cell printer. Second lead end 16 of coiled connector 12 features a metal sleeve 22, a PEEK insert 24 and a tip 26. Tip 26 deposits the live cells or other media in a microwell or upon a desired substrate, matrix. Tip 26 is typically comprised of ceramic.

In commercial embodiments, metal sleeve 22, PEEK insert 24, tip 26 and coiled section 18 of coiled connector 12 exist as independent components and are connected on both ends 14 16 within the cell printer during manufacture or service to allow for fluid transport. In this manner, should one of the components break and need to be replaced, the procedure requires taking apart all the pieces to locate the defective piece and replace it. Such a practice is time-consuming, expensive and can result in contamination.

The instant disclosure contemplates a unitary or singular component in which metal sleeve 22, PEEK insert 24, tip 26 and coiled section 18 (with its first and second ends 14, 16) all being integral with each other as a single component. In an alternative embodiment, the unitary component can be constructed without a PEEK insert 24. In accordance with the disclosure, the unitary component is connected within the printer via a valve. The valve may be a Lee valve, a piezo valve, or any other type of valve or suitable connection apparatus as would be evident to one having ordinary skill in the art.

4

The unitary component is intended to be a consumable. As such, repair or replacement requires removing the unitary component and replacing it with a new component. This saves time and reduces contamination since repair time is reduced and fewer parts are handled.

Metal sleeve 22 and PEEK insert 24 on first lead end 14 of coiled connector 12, as well as valve 20, are typically encompassed within a ferrule 28 (shown in FIG. 3). Ferrule 28 facilitates the connections on first end 14 and second end 16 of coiled connector 12 with other components of the cell printer (typically by screwing) and also facilitates easy identification of these parts.

In an alternative embodiment, coiled connector 12 may optionally eliminate PEEK insert 24, such that metal insert 22 connects directly with printer tip 26.

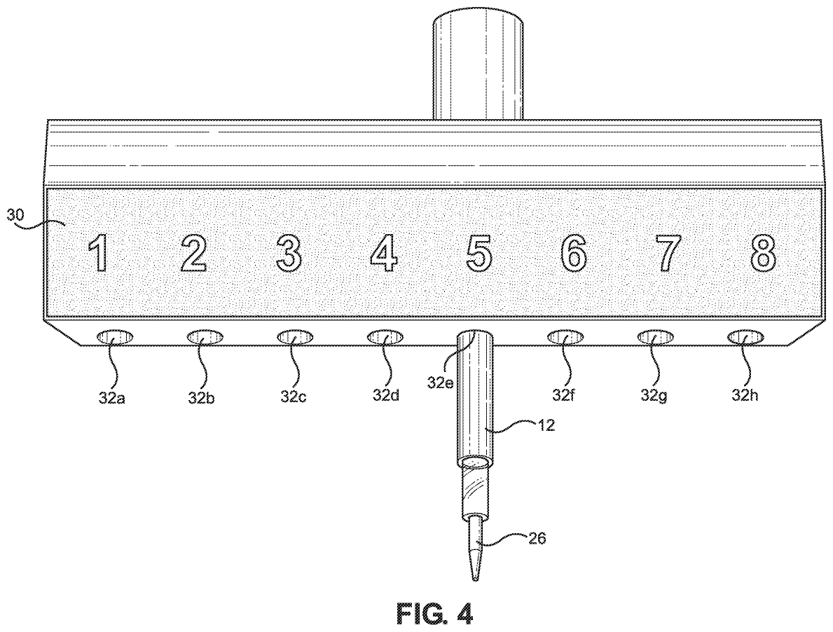

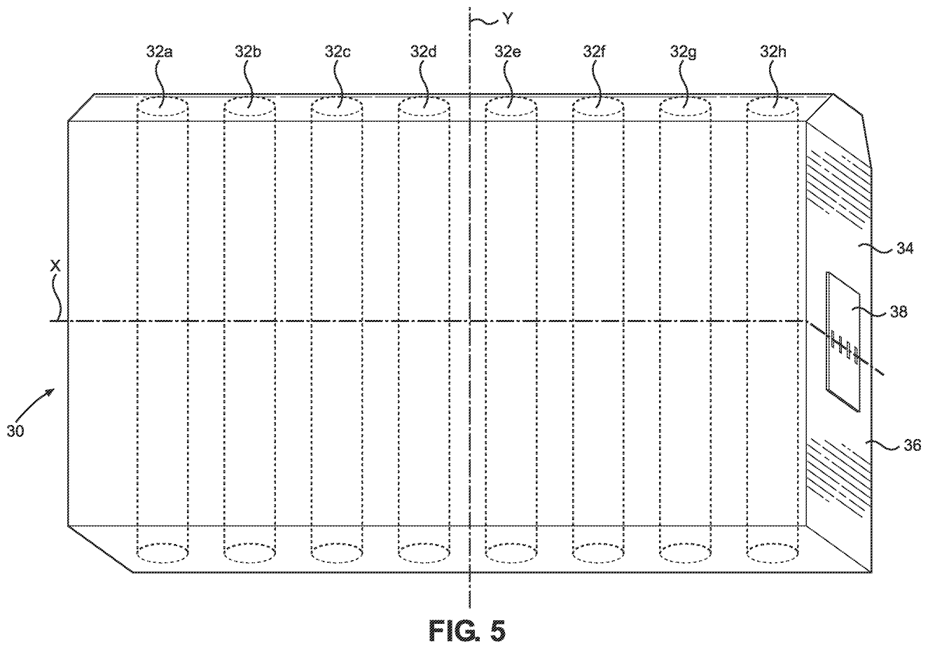

FIG. 4 shows a printer block 30 in accordance with the instant disclosure. In operation, printer block 30 is positioned within a cell printer and is configured with openings or bores (32a-32h) through which one or more unitary coiled connectors 12 in accordance with the instant disclosure are positioned. In the embodiment of FIG. 5, eight bores (32a-32h) are shown and are labeled 1-8 on the face of printer block 30. This disclosure is not limited in the number of bores within a printer block. FIG. 5 further illustrates a unitary coiled connector 12 disposed within bore number 5 of printer block 30.

It is essential in the printing operation that the one or more unitary coiled connectors 12 disposed within the bores in printer block 30 are maintained in a rigid manner so that it is secured during the printing process to prevent extraneous movement of coiled connectors 12 while cells are being printed. Additionally, if a specific unitary coiled connector needs to be removed or replaced, it is essential that other unitary coiled connectors in the printer block not be disturbed. To avoid any movement of the one or more unitary coiled connectors, the instant disclosure, in one embodiment, such as illustrated in FIG. 6, contemplates a printer block having a sandwich-type configuration in which the printer block is divided or bisected, for example along the axis indicated by the letter X, and such that one portion of the block, for example, a lower portion of the block 36 retains the unitary coiled connectors in a secure manner and an upper portion of the block 34 is configured to swing open, such as via a hinge 38 (and as indicated by arrow Y in FIG. 5), in order to provide access to the unitary coiled connectors disposed within the bores of the printer block. In this manner, the upper portion of the printer block can be opened and any unitary coiled connector that needs to be added, removed, or replaced. Once the removal, replacement or repair has been made, the upper portion of the printer block will be returned to its original (closed) position and then secured by any known locking mechanism, such as, but not limited to, a clamp.

In an alternative embodiment of the printer block shown in FIG. 6, upper portion 34 may be halved, e.g., further bisected along its transverse axis, such that only a front portion of the upper portion 34 is swung open while the back half of the upper portion 34 remains stationary and is integral with the lower portion for holding the coiled connectors within the bores. In this embodiment, the printer block is not limited in the manner in which it is opened and subsequently secured. It is contemplated the printer block may be bisected along the axis indicated by the letter X, such that the upper portion is hinged to be lifted and opened (and subsequently lowered to be secured), or the block may comprise front and back portions such that the bores are secured to either the front or back portion wherein the front or back portion not comprising the bores may be hinged for opening and closing.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating an orientation (e.g., “top”, “bottom”, “side”, etc.) is for convenience of description and does not require any particular orientation of the item described.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described. In the claims the words “including”, “carrying”, “having”, and the like mean, as “comprising”, including but not limited to.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A coiled connector component for use in a cell printer, the coiled connector component having a first end and a second end, and comprising:
1. a coil section;
2. a sleeve;
3. a PEEK insert; and
4. a printer tip,
wherein the coil, the sleeve, the PEEK insert and the printer tip comprise a unitary component.

2. The coiled connector component according to claim 1, further comprising a ferrule.

3. The coiled connector component according to claim 2, wherein a ferrule is positioned on the first end and the second end of the connector component.

4. The coiled connector component according to claim 1, wherein the coil section is connected within the cell printer via a valve selected from the group consisting of a Lee valve, a piezo valve or any suitable connection apparatus.

5. The coiled connector component according to claim 4, wherein the valve is a Lee valve.

6. The coiled connector component according to claim 4, wherein the valve is a piezo valve.

7. The coiled connector component according to claim 4, wherein the valve is disposed within the printer.

8. The coiled connector component according to claim 1, wherein the coil section is selected from an inert material acceptable for use in medical devices according to the U.S. Food & Drug Administration (FDA).

9. The coiled connector component according to claim 8, wherein the coil is comprised of a material made of plastic.

10. The coiled connector component according to claim 1, wherein the sleeve is metal.

11. The coiled connector component according to claim 1, wherein the printer tip is ceramic.

12. The coiled connector component according to claim 1, configured to be positioned within a printer block.

13. A cell printer block for holding one or more cell printer tips, the cell printer block having a front face, a rear face, a top portion and a bottom portion, and wherein the block has one or more bores extending through the block from the top portion through the block and exiting the bottom portion, each of the bores configured to receive and retain a printer tip therein.

14. The cell printer block according to claim 13, wherein the block is bisected to separate the top and bottom portion.

15. The cell printer block according to claim 14, wherein the top and bottom portions are secured to one another via a hinge or clamp.

16. The cell printer block according to claim 13, wherein the printer tip is ceramic.

17. The cell printer block according to claim 13, wherein the one or more printer tips are part of one or more coiled connector components, the coiled connector components further comprising a coil section, a sleeve and a PEEK insert integral with the one or more printer tips.

18. The cell printer block according to claim 17, wherein the wherein the coil section, the sleeve, the PEEK insert and the printer tip comprise a unitary component.

19. A coiled connector component for use in a cell printer, the coiled connector component having a first end and a second end, and comprising:
a. a coil section;
b. a sleeve; and
c. a printer tip,
wherein the coil, the sleeve, and the printer tip comprise a unitary component.

20. The coiled connector component according to claim 19, wherein the coiled connector is configured to be positioned within a printer block, and wherein the coil section is connected within the cell printer via a valve selected from the group consisting of a Lee valve, a piezo valve or any suitable connection apparatus.
